(19) United States
(12) Patent Application Publication
(10) Pub. No.: US 2007/0225325 A1
(43) Pub. Date: Sep. 27, 2007

(54) Solid Forms of Montelukast Acid

(75) Inventors: Ernest Mestrovic, Bjelovar (HR); Michaela Horvat, Sesvete (HR); Maja Devcic, Pozega (HR); Amir Avdagic, Zagreb (HR); Dominik Cincic, Zagreb (HR); Marina Marinkovic, Sesvete (HR)

Correspondence Address: RISSMAN JOBSE HENDRICKS & OLIVERIO, LLP, ONE STATE STREET, SUITE 800, BOSTON, MA, 02109, US

(73) Assignee: PLIVA-ISTRAZIVANJE I RAZVOJ D.O.O., Zagreb (HR)

(21) Appl. No.: 10/587,537

(22) PCT Filed: Jan. 19, 2005
(86) PCT No.: PCT/HR05/00005

§ 371 Date: Jun. 7, 2007

Related U.S. Application Data
(60) Provisional application No. 60/540,307, filed on Jan. 28, 2004.

(51) Int. Cl. C07D 215/00

(52) U.S. Cl. 514/311; 546/180

(57) ABSTRACT

Differing forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid and methods for synthesizing same.

### FIELD OF THE INVENTION

The present invention relates to a new crystalline and new amorphous forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid (“montelukast acid”), to processes for their preparation, to pharmaceutical formulations containing them, and to a method of treatment using the same.

### BACKGROUND OF THE INVENTION

The sodium salt of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid (Montelukast sodium) is a therapeutic agent useful for the treatment of bronchial asthma. Montelukast sodium is disclosed in European Patent Application No. 480,717 the disclosure of which is enclosed here by reference.

The EP 480,717 does not disclose the solid state characterization of 1-(((1(R)-(3-(2-(7-chloro-2-quinolinyl)ethenyl)-phenyl)-3-(2-(1-hydroxy-1 methylethyl)phenyl)propyl)thio)methyl)cyclopropane acetic acid or the sodium salt thereof. Since various polymorphic forms of the same substance can have different characteristics which are important factors when this substance is used in pharmaceutical formulations, it was an object to find and characterize different solid states of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid and their methods of production.

### SUMMARY OF THE INVENTION

In one embodiment, the present invention is directed to a new crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

In a second embodiment, the disclosure is directed to a process for the preparation of the new crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

A further embodiment relates to new amorphous forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid as well as methods for their preparation.

A further embodiment is directed to a pharmaceutical composition containing the new crystalline and amorphous forms, and yet another embodiment is directed to a method of treatment using the new crystalline and amorphous forms.

### BRIEF DESCRIPTION OF THE DRAWINGS

FIG. 1 shows an XRPD pattern of the crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 2 shows an IR spectrum of the crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 3 shows a DSC thermogram of the crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 4 shows an XRPD pattern of amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 5 shows an IR spectrum of amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 6 shows a DSC thermogram of amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 7 shows an XRPD pattern of amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 8 shows an IR spectrum of amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

FIG. 9 shows a DSC thermogram of amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

### DETAILED DESCRIPTION OF THE INVENTION

A new and inventive crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid (“montelukast acid”) having characteristic X-ray powder diffraction peaks, designated by 2θ and expressed in degrees, at 6.5±0.2°, 10.0±0.2°, 15.5±0.2°, 18.3±0.2°, 20.4±0.2° and 24.6±0.2° has been defined. This crystalline form of montelukast acid has shown to have a higher stability than the montelukast sodium and doesn't absorb water compared to the sodium salt so that its formulation is easier. Furthermore, the production of the crystalline form of montelukast acid is simpler than that of the sodium salt since it requires fewer production steps.

Preferably, the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid shows a monoclinic space group P 2₁ and displays unit cell parameters comprising: crystal axis lengths of a=7.95±0.02 Å, b=21.94±0.02 Å, c=17.95±0.02 Å and an angle between the crystal axes of β=100.0 3±0.02°. With this single crystal data the crystal form is clearly identified.

According to an advantageous embodiment, the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid is provided with a purity of greater than 90.0%, preferably greater than 95%, preferably greater than 99%, preferably greater than 99.9%. This highly chemically and polymorphically pure form of montelukast acid is useful for the production of pharmaceutical formulations.

According to a further aspect of the present invention, a process for preparing the above defined, inventive crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid is provided, comprising the steps

dissolving a salt of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid in a solution A comprising at least one organic solvent,

converting the salt of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid into acid,

crystallizing the 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid, and

optionally isolating the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

Preferably said dissolving, converting and/or crystallizing step/steps is/are carried out by mixing the solution, for example by stirring or shaking. Still preferred the solution A comprising the montelukast acetic acid or salt thereof is sonicated in order to enhance the dissolving, converting and/or crystallizing.

Advantageously a sodium salt of montelukast is dissolved, since this salt has shown excellent dissolution characteristics. Furthermore, the starting material used to prepare the new crystalline form may be any amorphous or crystalline form of montelukast acetic acid or a salt thereof.

Furthermore, two or more organic solvents may be present in said solution A. Preferably, said at least one organic solvent is selected from the group consisting of ketons, esters, alcohols and halogenated solvents. Still preferred said at least one organic solvent is acetone which induces crystal formation of the montelukast acetic acid quickly and with high yield of the pure polymorph.

The converting step is only necessary in the case the montelukast salt is dissolved, whereby it can be carried out simultaneously with the dissolving step and/or the crystallizing step. Methods for converting a salt of a given substance into its acid are well known to the person skilled in the art and any conventional method can be used.

For the crystallization, which defines the step in which the crystals of the montelukast acetic acid are formed, the pH range of the solution is typically between 1 and 8, preferably from 3 to 7, and most preferably from 4 to 6. The crystallization temperature is preferably between 5° C. and 50° C., still preferred from 10° C. to 40° C., and most preferred from 20° C. to 30° C.

In general, conversion and crystallization are carried out in one reaction step which simplifies the production process.

The isolating step comprises preferably the filtering of the crystals, and the heating of the solution in order to evaporate the solution, respectively. The heating is advantageously carried out under vacuum to dryness. The aim of the isolating step is to provide dried and pure montelukast acid crystals according to the present invention which can then be used for further processing, e.g. the production of a pharmaceutical formulation.

Preferably, the converting step is carried out with a solution B comprising at least one aqueous solution and a chromatographic column, respectively. The at least one aqueous solution can be any solution known in the state of the art which is used for converting a salt into an acid. This is preferably a solution with a pH of under 7. Therefore, the aqueous solution preferably shows a specific pH depending on the salt. Of course, the solution B can comprise any further ingredients useful for inducing and enhancing the converting step. In case solutions A and B are provided at the same time, e.g. in order for the converting, the dissolution and crystallizing steps to be carried out simultaneously, the ratio of solution B to solution A used is advantageously from about 1:10 to about 10:1, preferably from about 1:4 to about 4:1 and most preferably from about 1:2 to about 2:1 (v:v).

According to a preferred embodiment, the at least one aqueous solution is selected from the group consisting of hydrochloric acid, citric buffer, acetate buffer, biphtalate buffer, and phosphate buffer. These aqueous solutions have shown to enhance the converting of montelukast salt into montelukast acid in an optimal way.

According to a particularly advantageous embodiment, the montelukast acid is eluted from the chromatographic column with said solution A comprising at least one organic solvent. Preferably said solution A comprises two organic solvents, preferably ethyl acetate and methylene chloride, preferably at a ratio between 1:1 and 10:1, preferably 4:1 (v:v). This specific solution A induces the formation of the crystal in a particularly optimal way.

According to this process, the column is charged preferably with a solution comprising the salt of montelukast, still preferred the sodium salt, and the column is selected so as to induce conversion of the salt into the acid. A preferred filling of the column is therefore a ion exchanger, silica gel or aluminium oxide.

A further aspect of the present invention relates to an amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid having a characteristic DSC thermogram with two endothermic peaks, one at between 43° C. and 53° C., preferably between 47° C. and 49° C., preferably at 48° C. and one at between 143° C. and 153° C., preferably between 147° C. and 149° C., preferably at 148° C. and further having one exothermic peak at between 86° C. and 96° C., preferably between 90° C. and 92° C., preferably at 91° C.

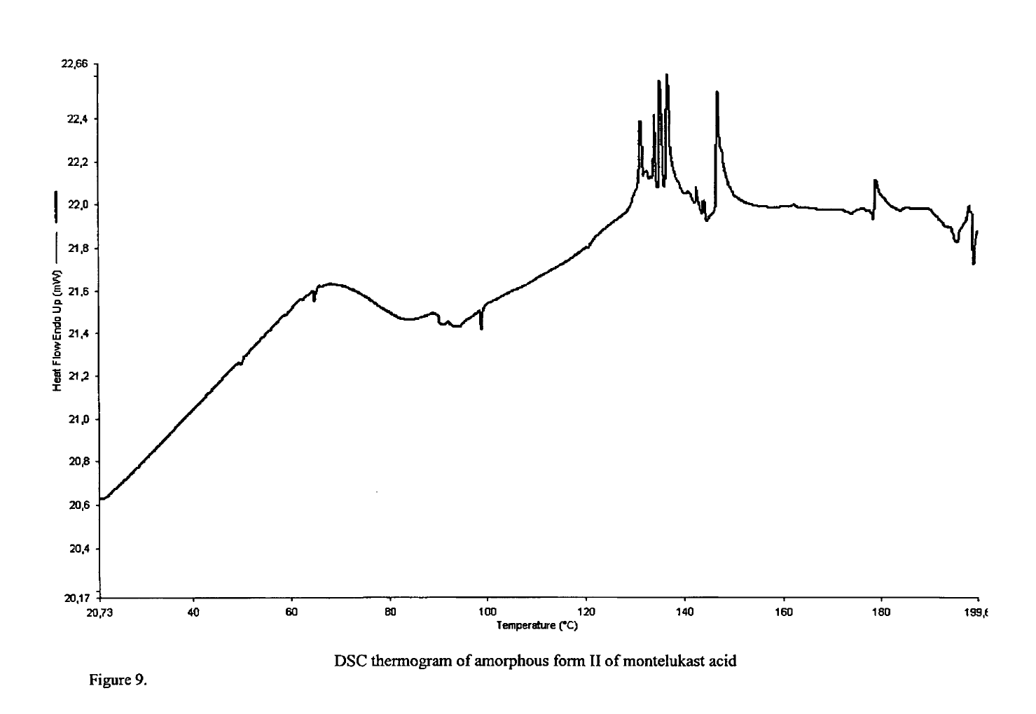

A further aspect of the present invention relates to an amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid. This amorphous form II of montelukast acid is defined as having a characteristic DSC thermogram as shown in FIG. 9 of the present application.

The amorphous forms show excellent solubility, e.g. faster dissolution, and therefore a better bioavailability. Furthermore, the amorphous forms show advantages with respect to free flowability and can easily be filtered. Since not every crystal form of a substance also has a corresponding amorphous form, it was surprising to provide an amorphous form of the montelukast acetic acid.

A further preferred aspect of the present invention is furthermore a process for preparing the amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid as defined above, comprising grinding the crystal form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid. The grinding step is preferably carried out in a suitable mill, advantageously at 100 to 1000 rpm, preferably at 700 to 900 rpm. Advantageously said grinding step is carried out for 30 min to 2 h, preferably for 1 h.

According to a further aspect of the present invention, a process for preparing the amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid is provided comprising
i) providing a suspension of the crystal form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid, or a salt thereof, in an acidic aqueous solution and
ii) isolating said amorphous form II.
   Preferably said salt is the sodium salt since this is particularly useful for the preparation of the above amorphous form.
   Said acidic aqueous solution has a pH of under 7. Still preferred, the acidic aqueous solution is a solution comprising a mineral acid, said solution preferably having a pH of ≦3, preferably a pH of ≦2, still preferred a pH of ≦1. Advantageously said aqueous solution comprises hydrochloride acid.

Again, it is preferred that the suspension is mixed, for example shaken or stirred. During this period which optimally lasts 30 min to 24 h, preferably 1 h, the amorphous form II is produced, whereby a temperature of 10-40° C., preferably 20-25° C., is particularly advantageous. Again, the isolation is preferably carried out by filtration and evaporating said aqueous solution, respectively, for example under vacuum and at a temperature of between 20 and 80° C., preferably at 50° C.

In all the above described processes, the produced montelukast acid is preferably washed and dried in order to provide the pure crystal and amorphous forms, respectively.

According to a further advantageous embodiment, a pharmaceutical composition comprising the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid, the amorphous form I, and/or the amorphous form II is provided and one or more pharmaceutically acceptable carriers or excipients.

The new crystalline and amorphous forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid of the present invention can be utilized in the preparation of rapid, controlled and sustained release pharmaceutical formulations, suitable for oral, rectal, parenteral, transdermal, buccal, nasal, sublingual, subcutaneous or intravenous administration. Such formulations may be useful for the treatment of asthma in a human. Therefore, a further preferred embodiment of the present invention is the crystalline, amorphous form I and/or amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid for the use of treating asthma in a human as well as a method for treating asthma in a human whereby said pharmaceutical composition as defined above is administered to said human.

The formulations are preferably administered orally, in the form of rapid or controlled release tablets, microparticles, mini tablets, capsules and oral solutions or suspensions, or powders for the preparation thereof. In addition to one or more of the new forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid of the present invention as the active substance, oral preparations may optionally include various standard pharmaceutical carriers and excipients, such as binders, fillers, buffers, lubricants, glidants, disintegrants, odorants, sweeteners, surfactants and coatings. Some excipients may have multiple roles in the formulations, e.g., act as both binders and disintegrants.

Examples of pharmaceutically acceptable disintegrants for oral formulations useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral formulations useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral formulations include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the formulations of the invention include, but are not limited to, magnesium stearate, talc, polyethylene glycol, polymers of ethylene oxide, sodium lauryl sulfate, magnesium lauryl sulfate, sodium oleate, sodium stearyl fumarate, DL-leucine and colloidal silicon dioxide

Examples of suitable pharmaceutically acceptable odorants for the oral formulations include, but are not limited to, synthetic aromas and natural aromatic oils such as extracts of oils, flowers, fruits and combinations thereof. Preferable are vanilla and fruit aromas, including banana, apple, sour cherry, peach and similar aromas. Their use depends on many factors, the most important being the organoleptic acceptability for the population that will be taking the pharmaceutical formulations.

Examples of suitable pharmaceutically acceptable dyes for the oral formulations include, but are not limited to, synthetic and natural dyes such as titanium dioxide, beta-carotene and extracts of grapefruit peel.

Examples of useful pharmaceutically acceptable coatings for the oral formulations, typically used to facilitate swallowing, modify the release properties, improve the appearance, and/or mask the taste of the formulations include, but are not limited to, hydroxypropylmethylcellulose, hydroxypropylcellulose and acrylate-methacrylate copolymers.

Suitable examples of pharmaceutically acceptable sweeteners for the oral formulations include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Formulations of the new forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid of the present invention can also be administered intravenously or intraperitoneally, by infusion or injection. Dispersions can also be prepared in a liquid carrier or intermediate, such as glycerin, liquid polyethylene glycols, triacetin oils, and mixtures thereof. To improve storage stability, such preparations may also contain a preservative to prevent the growth of microorganisms.

Pharmaceutical formulations suitable for injection or infusion may be in the form of a sterile aqueous solution, a dispersion or a sterile powder that contains the active ingredient, adjusted, if necessary, for preparation of such a sterile solution or dispersion suitable for infusion or injection. This may optionally be encapsulated into liposomes. In all cases, the final preparation must be sterile, liquid, and stable under production and storage conditions.

The liquid carrier or intermediate can be a solvent or liquid dispersive medium that contains, for example, water, ethanol, a polyol (e.g. glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants. Prevention of the action of microorganisms can be achieved by the addition of various antibacterial and antifungal agents, e.g. paraben, chlorobutanol, or sorbic acid. In many cases isotonic substances are recommended, e.g. sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Sterile injectable solutions can be prepared by mixing at least one of the new solid forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid with an appropriate solvent and one or more of the aforementioned excipients, followed by sterile filtering. In the case of sterile powders suitable for use in the preparation of sterile injectable solutions, preferable preparation methods include drying in vacuum and lyophilization, which provide powdery mixtures of the isostructural pseudopolymorphs and desired excipients for subsequent preparation of sterile solutions.

The compound of the present invention may also be used for the preparation of locally acting, topical formulations. Such formulations may also contain other pharmaceutically acceptable excipients, such as polymers, oils, liquid carriers, surfactants, buffers, preservatives, stabilizers, antioxidants, moisturizers, emollients, colorants and odorants.

Examples of pharmaceutically acceptable polymers suitable for such topical formulations include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

Examples of suitable pharmaceutically acceptable oils which are so useful include but are not limited to, mineral oils, silicone oils, fatty acids, alcohols, and glycols.

Examples of suitable pharmaceutically acceptable liquid carriers include, but are not limited to, water, alcohols or glycols such as ethanol, isopropanol, propylene glycol, hexylene glycol, glycerol and polyethylene glycol, or mixtures thereof in which at least one of the new solid forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid is dissolved or dispersed, optionally with the addition of non-toxic anionic, cationic or non-ionic surfactants, and inorganic or organic buffers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetraacetic acid (EDTA), thiourea, tocopherol and butyl hydroxyanisole.

Suitable examples of pharmaceutically acceptable moisturizers include, but are not limited to, glycerine, sorbitol, urea and polyethylene glycol.

Suitable examples of pharmaceutically acceptable emollients include, but are not limited to, mineral oils, isopropyl myristate, and isopropyl palmitate.

The use of dyes and odorants in topical formulations of the present invention depends on many factors of which the most important is organoleptic acceptability to the population that will be using the pharmaceutical formulations.

The therapeutically acceptable quantity of at least one of the new solid forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid of the present invention administered varies, dependent on the selected compound, the mode of administration, treatment conditions, age and status of the patient or animal species, and is subject to the final decision of the physician, clinician or veterinary doctor monitoring the course of treatment.

Suitable oral and parenteral doses may vary within the range of from about 14.5 to about 286 μg per kg of body weight per day, preferably from about 29 to about 214 μg per kg of body weight and more preferably from about 58 to about 143 μg per kg of body weight per day. At least one of the new solid forms of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid may be formulated in a single dosage form that contains from about 1 to about 50 mg, preferably from about 2 to about 20 mg, and more desirably from about 4 to about 10 mg of the active substance per unit dose.

### EXAMPLES

The following Examples illustrate the invention, but are not limiting.

### Example 1

Production of the Crystalline Form of Montelukast Acid

The 1-(((1(R)-(3-(2-(7-chloro-2-quinolinyl)ethenyl)-phenyl)-3-(2-(1-hydroxy-1-methylethyl)phenyl)propyl)thio)methyl)cyclopropane sodium salt (1 g) was suspended in acetone (100 ml) and citric buffer pH=5 (100 ml). The suspension was sonicated in ultrasound bath at a temperature of 20° C. for 3 minutes. The precipitant is formed after 24 h. The crystals were filtered off and dried at room temperature under atmospheric pressure to constant weight (0.85 g crystal form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid. A single crystal of the new crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid was prepared, and single crystal X-ray diffraction data collected using a Bruker Nonius FR591/Kappa CCD diffractometer using CuKα radiation. Pertinent crystallographic data thus obtained are set forth in Table 1.

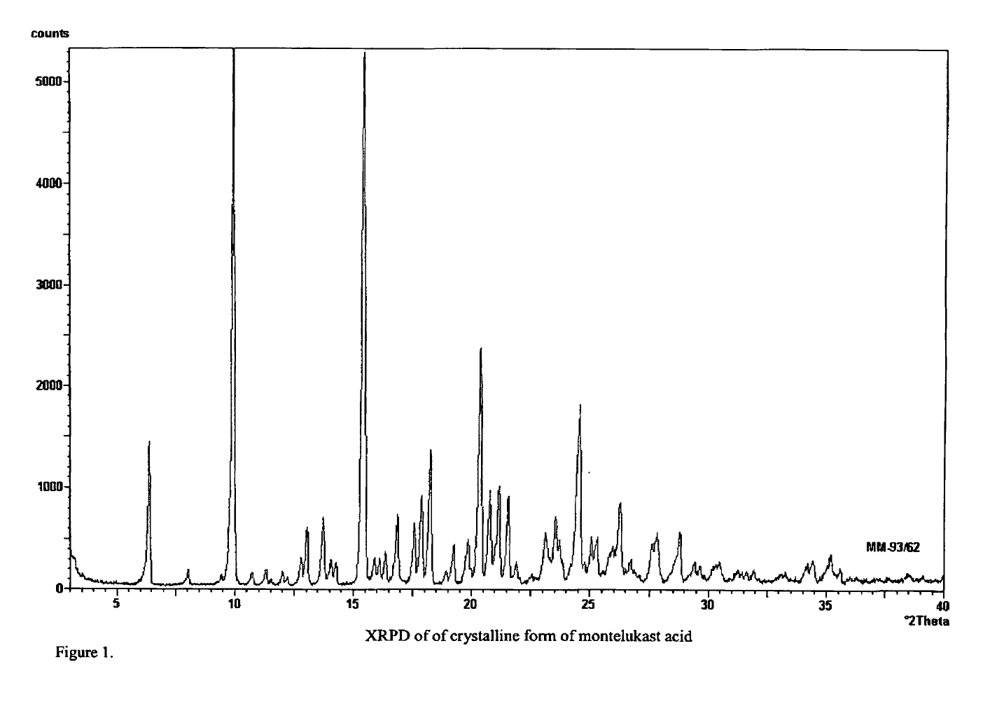

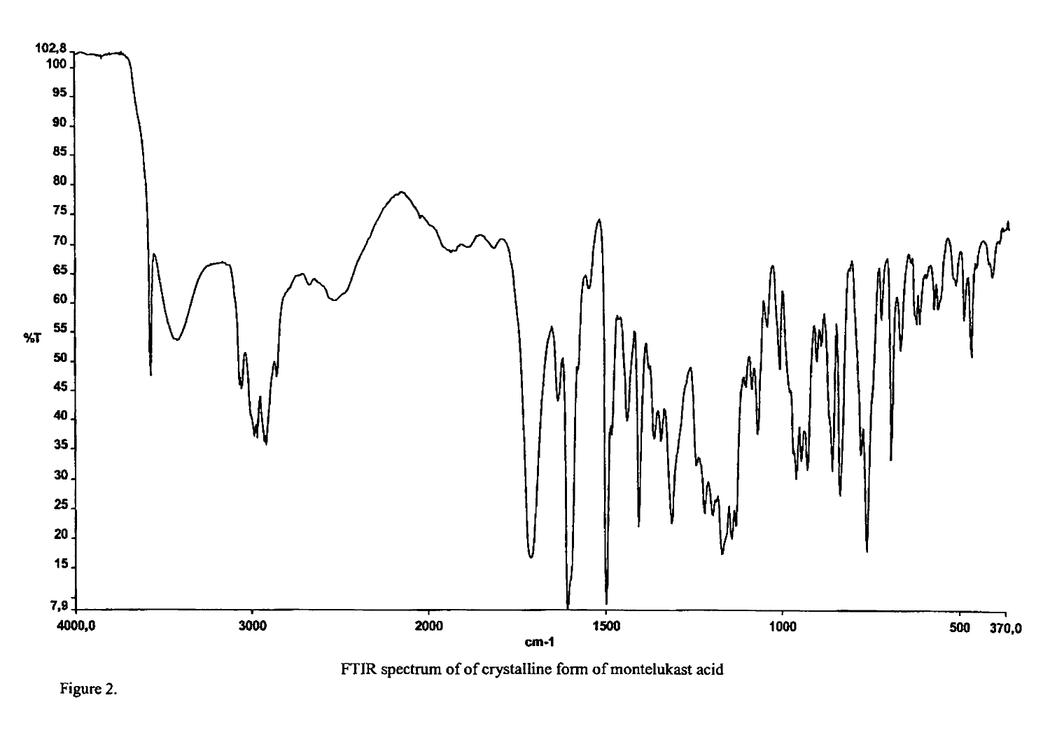

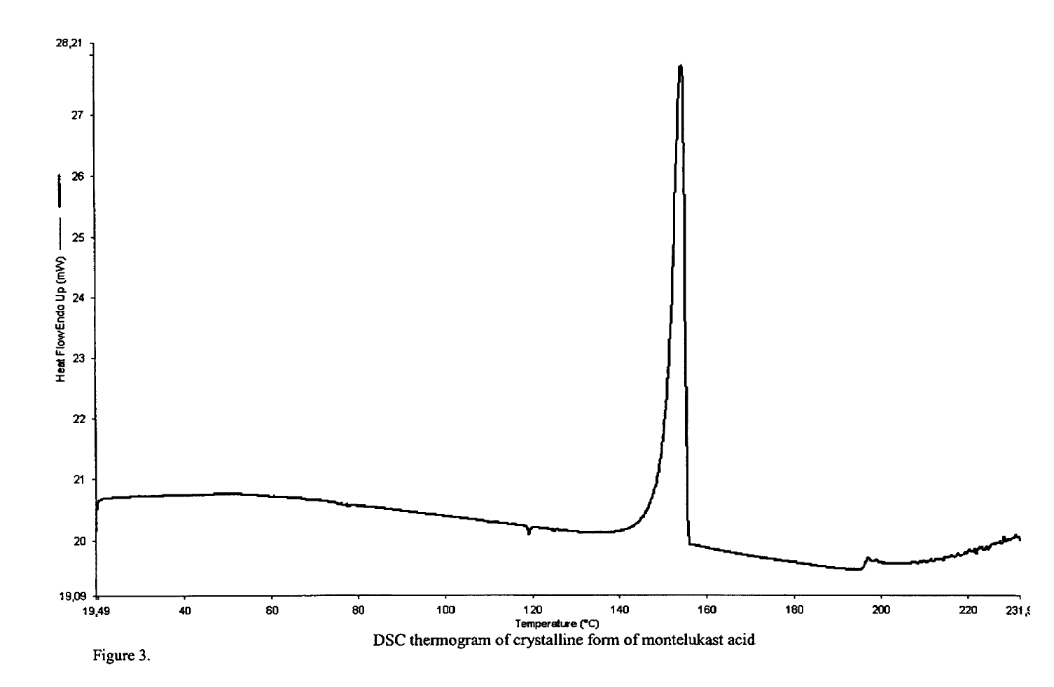

The new crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid was further characterized by x-ray powder diffraction (XRPD), Differential Scanning Calorimetry (DSC), and Infrared (IR) spectroscopy. The resulting XRPD pattern is shown in FIG. 1 where characteristic diffraction peaks, designated by 2θ and expressed in degrees, are detected at 6.5±0.2°, 10.0±0.2°, 15.5±0.2°, 18.3±0.2°, 20.4±0.2° and 24.6±0.2°. In the IR spectrum (see FIG. 2), characteristic bands are observed at 1715±5 cm⁻¹ and 3573±5 cm⁻¹. In the DSC (see FIG. 3), a characteristic endothermic peak in range from 120° C. to 180° C. is observed.

TABLE 1
Crystallographic data for the new
crystalline form of 1-(((1(R)-(3-(2-(7-
chloro-2-quinolinyl)ethenyl)-phenyl)-3-(2-(1-hydroxy-1-
methylethyl)phenyl)propyl)thio)methyl)cyclopropane acetic acid

| | |
|---|---|
| Chemical formula | (C₃₅H₃₅ClNO₃S)₂ |
| Empirical formula weight | 585.15 |
| Temperature | 293 ± 2K |
| Crystal size | 0.06 × 0.10 × 0.25 mm |
| Crystal system, space group | Monoclinic, P 2₁ |
| Unit cell dimension | a = 7.95 ± 0.02 Å |
| | b = 21.94 ± 0.02 Å |
| | c = 17.95 ± 0.02 Å |
| | β = 100.03 ± 0.02° |
| V | 3082 ± 2 Å³ |
| Z | 2 |
| Calculated density | 1.26 ± 0.02 g cm⁻³ |

### Example 2

Production of Crystalline Form of Montelukast Acid

The 1-(((1(R)-(3-(2-(7-chloro-2-quinolinyl)ethenyl)-phenyl)-3-(2-(1-hydroxy-1-methylethyl)phenyl)propyl)thio)methyl)cyclopropane sodium salt (1 g) was dissolved in acetone (15 ml) and 1% hydrochloric acid (7 ml) was added dropwise. The reaction mixture was stirred for 2 hours at 20-25° C. A yellow suspension was obtained and crystals of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid were isolated by filtration. The obtained yield was 76%.

The new crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid was further characterized by XRPD, DSC and IR spectroscopy. The DSC, IR spectrum, and XRPD pattern were the same as the ones described in example 1.

### Example 3

Production of the Crystalline Form of Montelukast Acid

The 1-(((1(R)-(3-(2-(7-chloro-2-quinolinyl)ethenyl)-phenyl)-3-(2-(1-hydroxy-1-methylethyl)phenyl)propyl)thio)methyl)cyclopropane sodium salt (5.75 g) was dissolved in ethyl acetate/methylene chloride (4/1; v/v) mixture. The solution was accumulated on a preparative chromatography column with silica gel filling and was eluded from the column using ethyl acetate/methylene chloride (4/1; v/v) mixture. The fractions eluded between 300 ml and 900 ml were collected and combined. The solvent was removed at about 50° C. under vacuum to dryness. This process produced free crystals of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid (5.00 g).

The new crystalline form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid has been further characterized by XRPD, DSC, and IR spectroscopy. DSC, IR, XRPD were the same as the ones described in example 1.

### Example 4

Production of Amorphous Form I of Montelukast Acid

The crystal form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane montelukast acid was ground in a ball mill at 800 rpm for 1 hour.

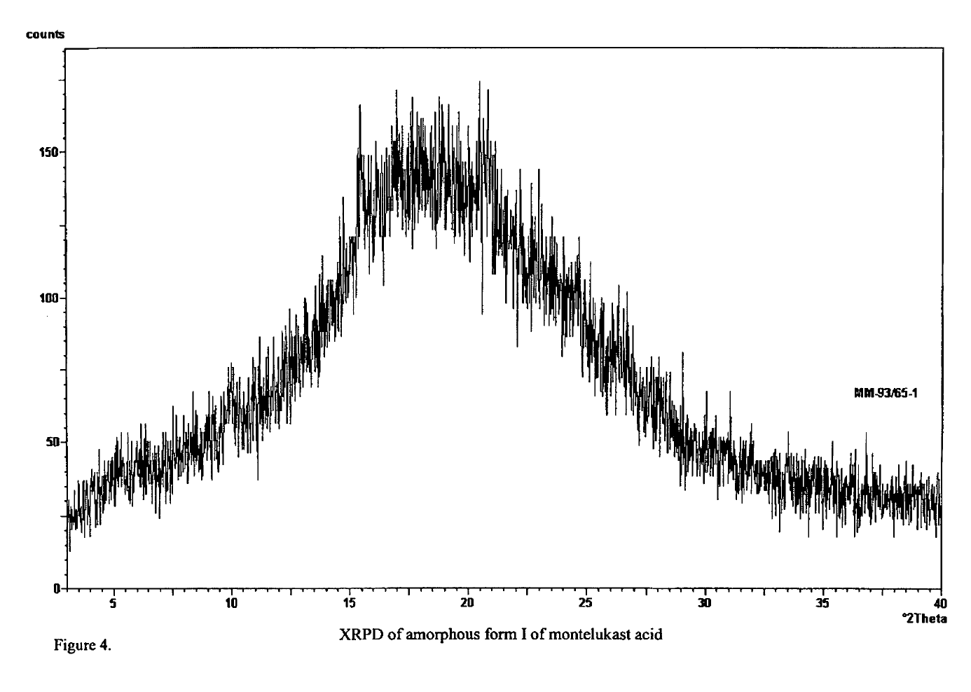

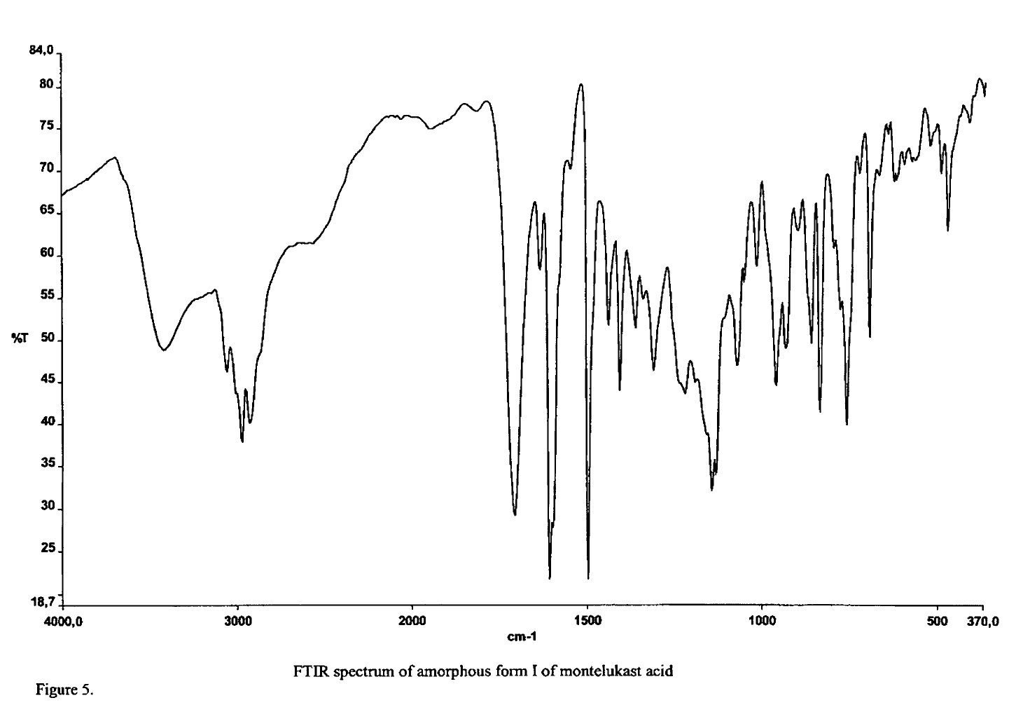

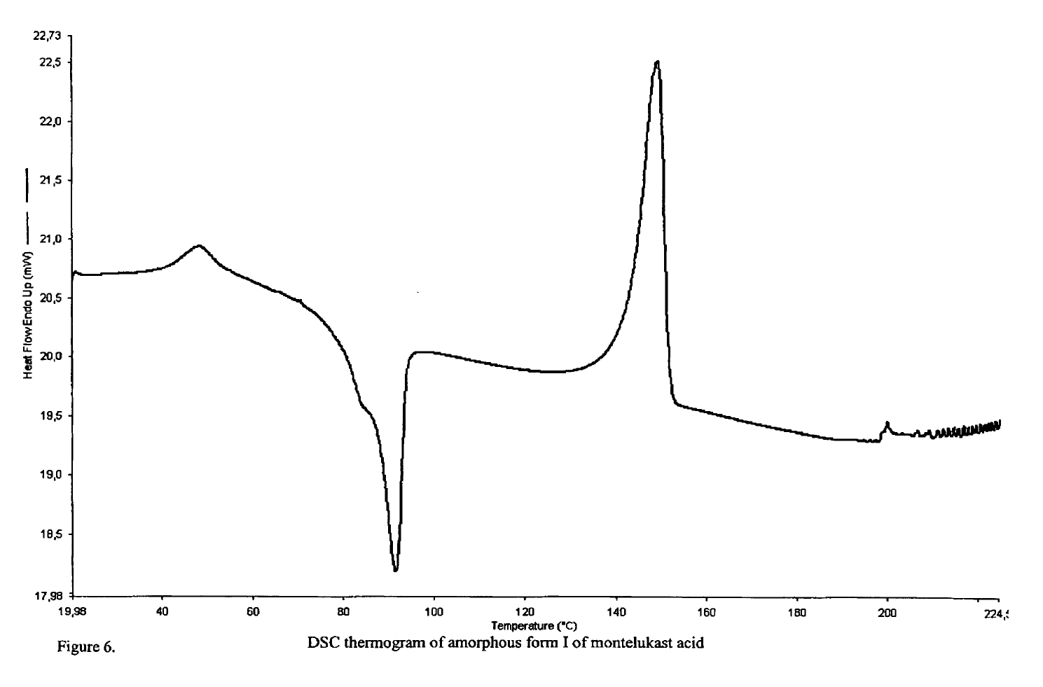

Amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane montelukast acid was prepared and characterized by XRPD, DSC, and IR spectroscopy. The XRPD pattern is shown in FIG. 4. In the IR spectrum (see FIG. 5), characteristic bands are observed at 1500, 1600, and 1700 cm⁻¹. In the DSC (see FIG. 6), a characteristic thermogram showed two endothermic peaks at about 48° C. and at about 148° C. and one exothermic peak at about 91° C.

### Example 5

Production of Amorphous Form II of Montelukast Acid

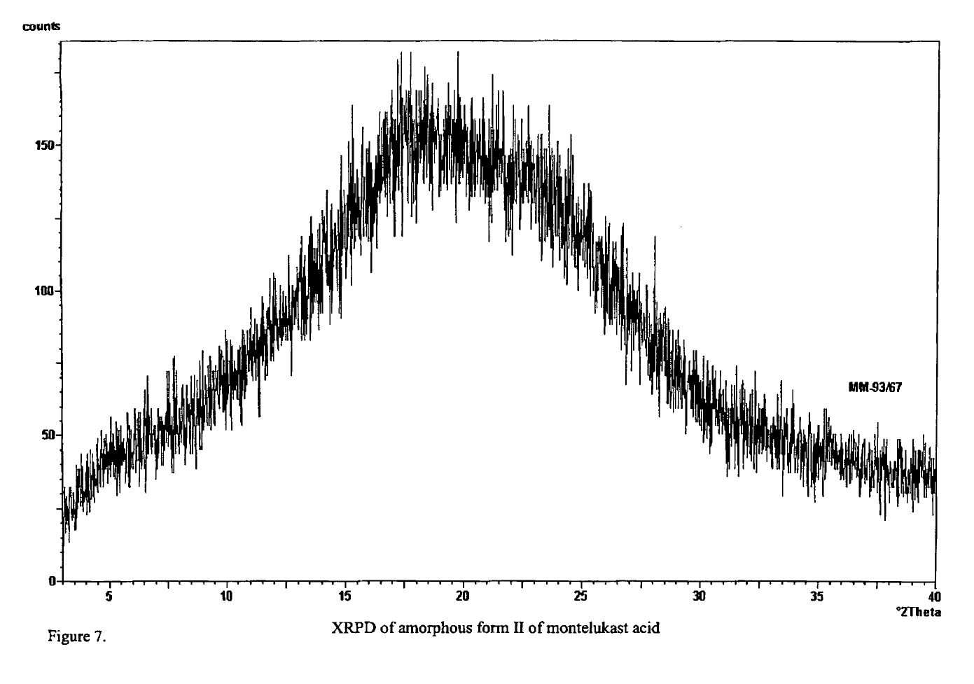

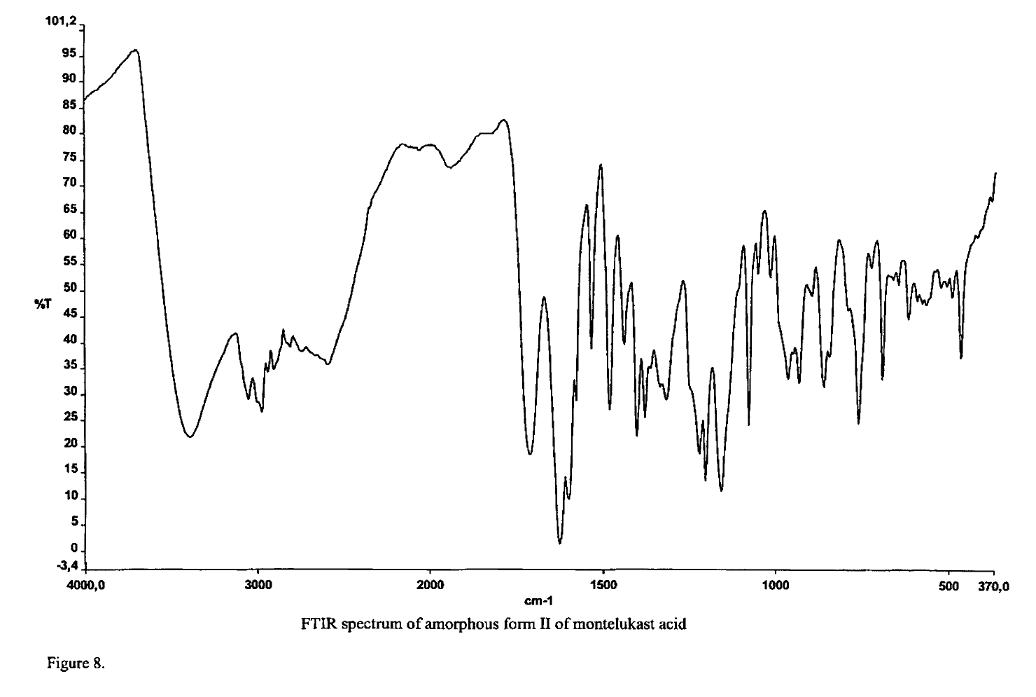

The 1-(((1(R)-(3-(2-(7-chloro-2-quinolinyl)ethenyl)-phenyl)-3-(2-(1-hydroxy-1-methylethyl)phenyl)propyl)thio)methyl)cyclopropane sodium salt (200 mg) was suspended in 1 M hydrochloric acid (3.5 ml) and stirred at 20-25° C. for 1 hour. Amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid was isolated by filtration, washed with water and dried on the air overnight with a resulting yield of 97%. This form was characterized by XRPD, DSC, and IR spectroscopy. The XRPD pattern is shown in FIG. 7, the IR spectrum is shown in FIG. 8, and the DSC is shown in FIG. 9.

1. Crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid having characteristic X-ray powder diffraction peaks, designated by 2θ and expressed in degrees, at 6.5±0.2°, 10.0±0.2°, 15.5±0.2°, 18.3±0.2°, 20.4±0.2° and 24.6±0.2°.

2. The crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to claim 1, characterized by a monoclinic space group P 2, and by displaying unit cell parameters comprising: crystal axis lengths of a=7.95±0.02 Å, b=21.94±0.02 Å, c=17.95±0.02 Å and an angle between the crystal axes of β=100.03±0.020.

3. The crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to claim 1, characterized in that it is provided with a purity of greater than 90.0%, preferably greater than 95%, preferably greater than 99%, preferably greater than 99.9%.

4. A process for preparing the crystalline 1-(((1(R)-(3-(2-(7-chloro-2-quinolinyl)ethenyl)phenyl)-3-(2-(1-hydroxy-1-methylethyl)phenyl)propyl)thio)methyl)cyclopropane acetic acid according to claim 1, comprising the steps
dissolving a salt of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid in a solution A comprising at least one organic solvent,
converting the salt of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid into acid,
crystallizing the 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid, and
optionally isolating the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

5. The process according to claim 4, characterized in that the converting step is carried out with a solution B comprising at least one aqueous solution and a chromatographic column, respectively.

6. The process according to claim 5, characterized in that the dissolving step and converting step are carried out together in a mixture comprising solution A and solution B, preferably in a ratio solution B:solution A of 1:10 to 10:1.

7. A process according to claim 5, characterized in that the 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid is eluted from the column with the solution A comprising at least one organic solvent.

8. Amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid having a characteristic DSC thermogram with two endothermic peaks, one at between 43° C. and 53° C., preferably between 47° C. and 49° C., preferably at 48° C. and one at between 143° C. and 153° C., preferably between 147° C. and 149° C., preferably at 148° C. and further having one exothermic peak at between 86° C. and 96° C., preferably between 90° C. and 92° C., preferably at 91° C.

9. A process for preparing the amorphous form I of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to claim 8, comprising grinding the crystal form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid.

10. Amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid having a characteristic DSC thermogram as shown in FIG. 9.

11. The process for preparing the amorphous form II of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to claim 10, comprising
providing a suspension of the crystal form of 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid, or a salt thereof, in an acidic aqueous solution and
isolating said amorphous form II.

12. A pharmaceutical composition comprising the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to claim 1, and one or more pharmaceutically acceptable carriers or excipients

13. Crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to claim 1, for the use of treating asthma in a human.

14. A pharmaceutical composition comprising the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to the amorphous form I of claim 8, and one or more pharmaceutically acceptable carriers or excipients.

15. A pharmaceutical composition comprising the crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to the amorphous form II of claim 10, and one or more pharmaceutically acceptable carriers or excipients.

16. Crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according the amorphous form I of claim 8, for the use of treating asthma in a human.

17. Crystalline 1-(((1(R)-(3-(2-(7-chloro-2-quinolinyl)ethenyl)-phenyl)-3-(2-(1-hydroxy-1-methylethyl)phenyl)propyl)thio)methyl)cyclopropane acetic acid according to the amorphous form II of claim 10, for the use of treating asthma in a human.
